(19) United States
(12) Patent Application Publication
(10) Pub. No.: US 2015/0250638 A1
(43) Pub. Date: Sep. 10, 2015

(54) CONTOURED ARM SLING

(71) Applicant: United Surgical Associates, Inc., Solvang, CA (US)

(72) Inventor: Mark E. Howard, Gardnerville, NV (US)

(21) Appl. No.: 14/639,648

(22) Filed: Mar. 5, 2015

Related U.S. Application Data
(60) Provisional application No. 61/949,976, filed on Mar. 7, 2014.

(51) Int. Cl. A61F 5/37

(52) U.S. Cl. CPC A61F 5/3738

(57) ABSTRACT

A sling for the arm incorporates first and second side panels each having a lower peripheral contour shaped to approximate a lower arm shape. An elastic bottom panel is attached to the first and second side panels at the lower peripheral contour.

### REFERENCE TO RELATED APPLICATIONS

This application claims priority of application Ser. No. 61/949,976 filed on Mar. 7, 2014 entitled Contoured Arm Sling and having a common assignee with the present application, the disclosure of which is incorporated herein by reference.

### BACKGROUND INFORMATION

1. Field

Embodiments of the disclosure relate generally to the field of anatomical support slings and more particularly to a sling for the arm which has a contoured lower periphery approximating the contour of a lower arm.

2. Background

Slings for support of an injured arm typically consist of a single wrapped sheet of fabric with an attached strap. While functional, these devices do not provide optimal support for the lower arm and may be uncomfortable when worn for extended periods. Traditional envelope arm slings can cause uneven distribution of forces that result in excessive pressure or “hotspots” on the forearm.

It is therefore desirable to provide an arm sling which provides a contour substantially matching the contour of the lower arm in a horizontal position thereby allowing a comfortable and fully supportive sling.

### SUMMARY

Embodiments disclosed herein provide an arm sling incorporating first and second side panels each having a lower peripheral contour shaped to approximate a lower arm shape. An elastic bottom panel is attached to the first and second side panels at the lower peripheral contour.

The features, functions, and advantages that have been discussed can be achieved independently in various embodiments of the present disclosure or may be combined in yet other embodiments further details of which can be seen with reference to the following description and drawings.

### BRIEF DESCRIPTION OF THE DRAWINGS

FIG. 1 is a side view of a pattern for the side panels of the sling;

FIG. 2 is a bottom view of a pattern for the bottom panel of the sling;

FIG. 3 is a pictorial view of the sling as assembled and applied to an arm for support showing the strap attachment and securing tabs; and,

FIG. 4 is a pictorial view of the sides and strap of the sling without the attachment and securing tabs for clarity.

### DETAILED DESCRIPTION

Embodiments disclosed herein provide a sling for the arm which has a contoured lower periphery approximating the contour of a lower arm.

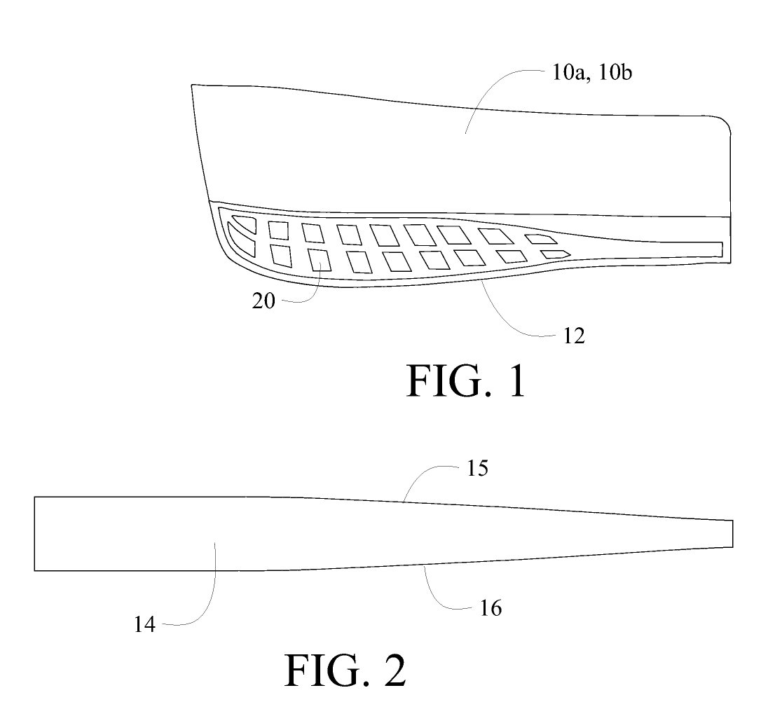

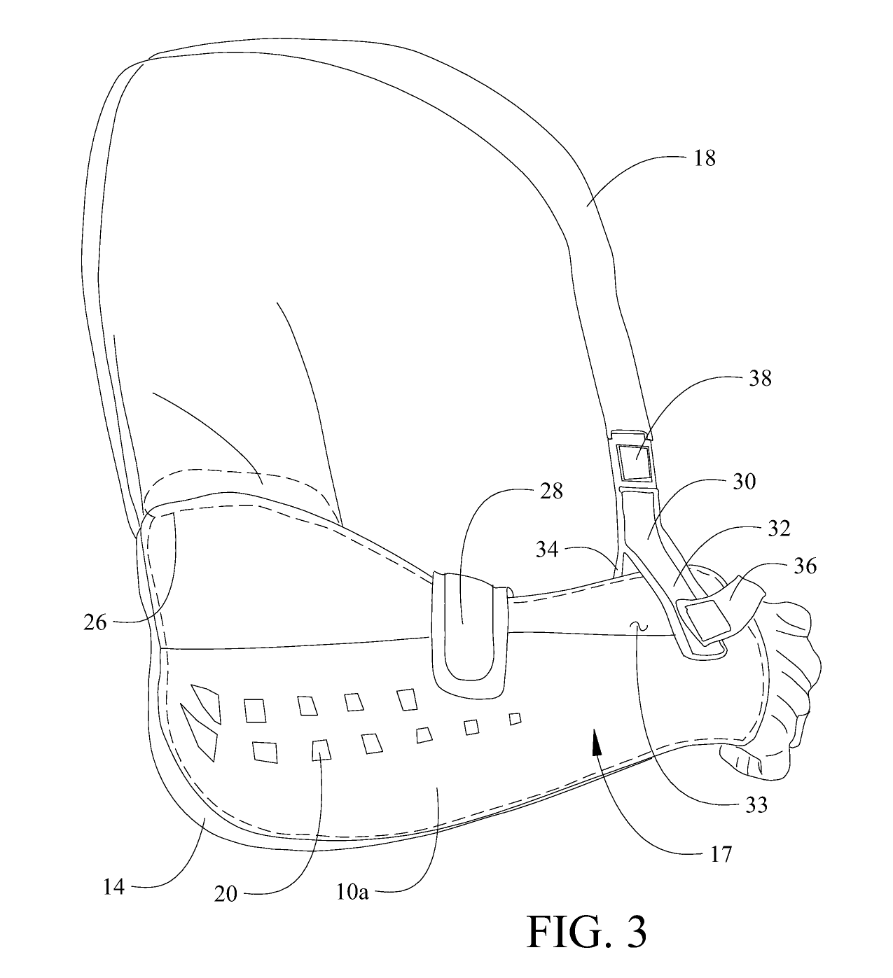

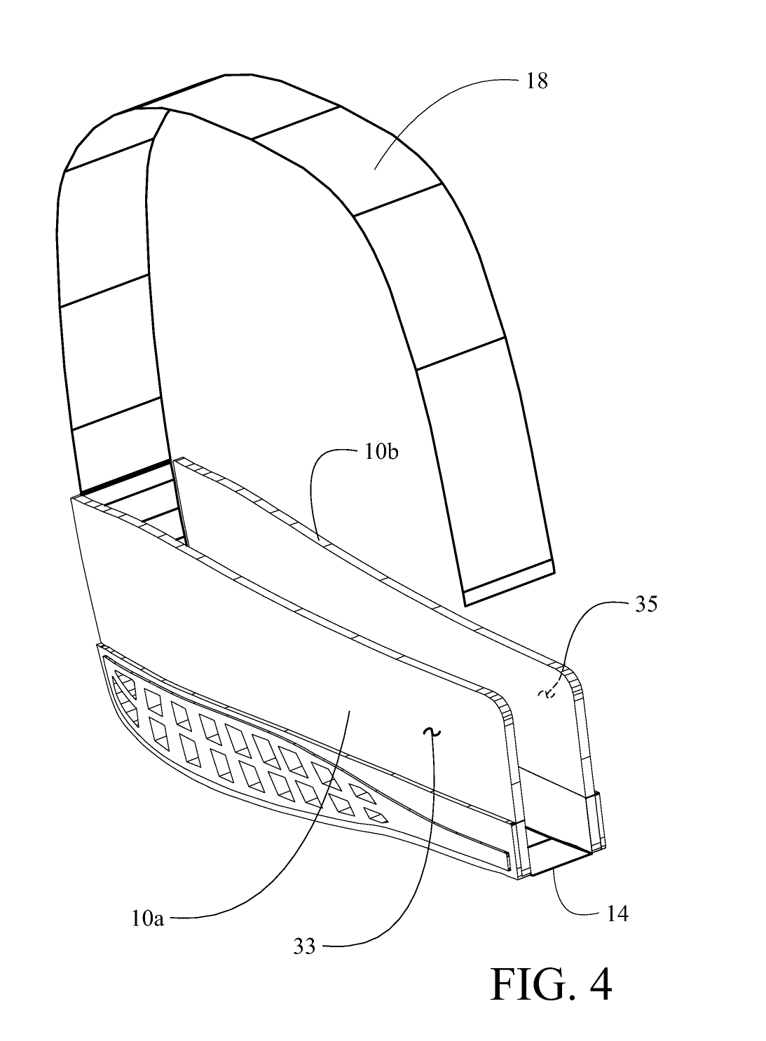

Referring to the drawings, as show in FIGS. 1 a pair of side panels, forward side panel 10a and rearward side panel 10b, have a lower peripheral contour 12 that is shaped to approximate the lower arm shape from elbow to wrist in a horizontal position. An elastic bottom panel 14 is shown in FIG. 2. The bottom panel 14 incorporates symmetrically tapering sides 15, 16. The bottom panel 14 is stitched or otherwise fastened to the side panels forming a sling 17 as shown in FIGS. 3 and 4 which then has the contoured supporting shape for the forearm with an elastic bottom contour to adapt to and support the bottom of the forearm and a taper from posterior to anterior end approximating the narrowing shape of the forearm from elbow to wrist. The taper of sides 15 and 16 is equal for the embodiment shown but may be non-symmetrical. Additionally for the embodiment shown the bottom panel extends up the posterior periphery of the side panels closing the sling about the elbow. The anterior end of the sling is open to allow the hand to protrude as with conventional slings. A sling strap 18 is attached to the anterior and posterior top portions of one or both side panels. The sling strap 18 may attach at the posterior portion to the upper extent of the bottom panel as attached to the side panels. The side panels 10a, 10b may be fabricated from neoprene, Lycra, Jersey, and polyester, foam, spandex and/or various laminated combinations or other suitable flexible fabrics. As shown in FIGS. 1 and 3 apertures 20 may be introduced into the side panels 10a, 10b for ventilation of the arm in the sling. The fabric for the side panels 10a, 10b of the sling may incorporate multiple layers of varying texture and thickness such as a lower portion 22 having greater thickness and resiliency and an upper portion 24. A lining 26 may be inserted into the sling as a removable element allowing the lining to be removed and washed without the need for washing the entire sling.

As shown in FIG. 3, closure of the upper opening of the sling 17 may be accomplished using a closure band 28. The anterior connection of the sling strap 18 may be accomplished using a Y connector 30 having a resiliently upstanding first leg 32 attached to a front surface 33 of an anterior portion of forward side panel 10a (best seen in FIG. 4) and a second leg 34, which may also be resiliently upstanding, attached to a rear surface 35 of an anterior portion of the rearward side panel 10b (best seen in FIG. 4) with attachment accomplished by a hook and loop fastener to allow the Y connector 30 to additional act as a closure for the anterior end of the sling 17. Safety bands 36 may extend from the anterior opening of the side panel 10 to further secure the Y connector to the sling. The sling strap 18 may include a releasable buckle or snap 38 located proximate the Y connector to allow disconnection of the strap without removal of the Y connector. Legs 32 and 34 urge the Y connector 30 into an upstanding position to allow one handed attachment of the buckle or snap 38.

Having now described various embodiments of the disclosure in detail as required by the patent statutes, those skilled in the art will recognize modifications and substitutions to the specific embodiments disclosed herein. Such modifications are within the scope and intent of the present disclosure as defined in the following claims.

What is claimed is:

1. A sling for the arm comprising:
forward and rearward side panels each having a lower peripheral contour shaped to approximate a lower arm shape; and, a bottom panel attached to the first and second side panels at the lower a peripheral contour.

2. The sling as defined in claim 1 wherein the bottom panel has tapering sides.

3. The sling as defined in claim 2 wherein the tapering sides are symmetrical.

4. The sling as defined in claim 1 wherein the bottom panel is elastic.

5. The sling as defined in claim 1 wherein at least one of the side panels incorporates apertures for ventilation.

6. The sling as defined in claim 1 further comprising a strap connected at a first end to a posterior end of one or more of the side panels and bottom panel and at a second end to a Y connector, said Y connector connected with a first leg to a front surface of an anterior portion of the forward side panel and with a second leg to rear surface of an anterior portion of the rearward side panel.

7. The sling as defined in claim 1 further comprising a closure band releasably engaged from the forward side panel to the rearward side panel.

8. The sling as defined in claim 1 wherein at least one side panel incorporates multiple layers of varying texture and thickness including a lower portion having greater thickness and resiliency than an upper portion.

9. The sling as defined in claim 1 wherein the side panels are fabricated from material selected from the set of neoprene, Lycra, Jersey, polyester, foam, spandex and various laminated combinations thereof

10. The sling as defined in claim 6 wherein the first leg of the Y connector is resiliently upstanding.

11. The sling as defined in claim 10 wherein the second leg of the Y connector is resiliently upstanding.

12. The sling as defined in claim 6 wherein the second leg of the Y connector is attached to the rear surface of the rearward side panel by a hook and loop fastener to allow the Y connector to additionally act as a closure for the anterior end of the sling.
